(19) United States
(12) Patent Application Publication
(10) Pub. No.: US 2006/0148254 A1
(43) Pub. Date: Jul. 6, 2006

(54) Activated iridium oxide electrodes and methods for their fabrication

(76) Inventor: George Y. McLean, Menlo Park, CA (US)

Correspondence Address: VEDDER PRICE KAUFMAN & KAMMHOLZ, 222 N. LASALLE STREET, CHICAGO, IL, 60601, US

(21) Appl. No.: 11/031,669

(22) Filed: Jan. 5, 2005

(51) Int. Cl. H01L 21/30; H01L 21/4763; A61F 2/16

(52) U.S. Cl. 438/686; 438/650; 623/6.63

(57) ABSTRACT

A technique for activating iridium to produce iridium oxide is provided. In a device in which an iridium layer is electrically coupled to a semiconductor junction, a current is generated within the junction and an activation current is applied to the iridium layer via the conductive semiconductor junction. In a presently preferred embodiment, the junction current is generated via illumination of the semiconductor junction.

### FIELD OF THE INVENTION

The present invention relates generally to methods for oxidizing iridium electrode structures on semiconductor substrates, and to devices fabricated by such methods.

### BACKGROUND

Iridium oxide electrodes are useful in a wide variety of applications, including use as pH sensors and electrodes for neural stimulation. In their application to neural stimulation, iridium oxide electrodes offer the advantage of considerably larger surface charge capacity than bare metal electrodes, by permitting charge injection via reversible valence transitions between two stable oxide forms. Such iridium oxide electrodes can be formed by a variety of techniques, including sputter deposition, electroplating, thermal decomposition of iridium salts, and other methods.

A commercially-available technique for depositing iridium oxide is sputtering. Sputtering techniques have been used to deposit iridium oxide on semiconductor substrates, including doped silicon substrates. In such applications, electrical performance of the iridium oxide film depends in part upon the quality of the metal-to-semiconductor contact. One method for improving such metal-to-semiconductor contact is thermal annealing. However, thermal annealing is typically not used on iridium oxide films because the iridium oxide cannot tolerate the temperatures involved in the annealing process.

Another technique for forming iridium oxide involves the “activation” of elemental iridium. Activation of iridium comprises the formation of iridium oxide states via an electrochemical process. For example, iridium oxide electrodes may be formed by activation where an elemental iridium film is electrochemically oxidized to form the desired iridium oxide electrode. Such electrochemical activation can be achieved in a standard electrochemical cell including a reference electrode, a counter electrode, and a working electrode comprising or attached to the iridium. The working electrode is cycled between two extremes of electrochemical potential over many cycles to oxidize the iridium. Activation of elemental iridium offers at least two significant advantages over sputter deposition of iridium oxide. First, elemental iridium can withstand the temperatures required for annealing. Second, deposition of elemental iridium is more easily and consistently performed by systems designed specifically for the formation of high-quality metal-to-semiconductor interfaces.

While useful in many circumstances, electrochemical activation of iridium oxide electrodes is problematic in the absence of a means of making direct electrical contact with the electrodes, for example, when the electrodes are extremely small or numerous and electrically distinct, or are electrically isolated by semiconductor junctions within the substrate on which they are formed. In the absence of a means of making direct electrical contact with the iridium metal film, it becomes a practical necessity to control the potential of the film electrodes through the substrate, where the presence of semiconductor junctions makes control of the oxidation process more difficult.

Therefore, it would be desirable to provide improved methods to electrochemically activate iridium electrodes electrically coupled to one or more semiconductor junctions formed in a semiconductor substrate, where connection of a working electrode can be made through the bulk semiconductor substrate material.

### SUMMARY

The present invention provides for the fabrication of iridium oxide electrodes by oxidation of elemental iridium via an electrochemical activation process. In general, where an iridium layer is electrically coupled to a semiconductor junction, a current is generated within the junction so that the junction becomes sufficiently conductive to permit control over the iridium potential via the junction. Where the junction current is sufficiently greater in magnitude than the transient currents required to control the electrode potential, variations in the voltage across the semiconductor junction are sufficiently small to permit treatment of the voltage as a constant, thereby allowing the activation protocol to be adjusted simply by introducing a constant potential offset to account for the junction voltage. In a presently preferred embodiment, elemental iridium is deposited (typically by sputtering, electroplating, vapor deposition, or the like) in electrical communication with a semiconductor junction that is capable of producing a photocurrent when exposed to light. An activation current (sufficient to drive the electrode to the targeted electrochemical potentials) is delivered to the elemental iridium through the semiconductor junction to oxidize the iridium. While the activation current is being applied, the junction is illuminated to generate a photocurrent sufficiently larger in magnitude than the activation current to make variations in the voltage across the semiconductor junction insignificant.

Using the methods described herein, disadvantages of prior art activation techniques may be overcome, thereby opening the benefits of activation techniques to a wider array of devices. For example, using the methods of the present invention, relatively small implantable medical devices (preferably retinal prosthetic implants and/or retinal therapeutic implants) incorporating iridium oxide electrodes may be more effectively fabricated.

### BRIEF DESCRIPTION OF THE DRAWINGS

FIG. 1 is a cross-sectional view of a semiconductor substrate with iridium/iridium oxide electrodes formed over semiconductor junctions in accordance with the principles of the present invention.

FIG. 2 is a circuit diagram of an electrochemical cell useful for the oxidation of iridium in accordance with the principles of the present invention.

FIG. 3 is a schematic illustration of a cell for performing the iridium activation according to the methods of the present invention.

FIG. 4 is a chart illustrating the voltage applied between the reference electrode and the common terminal of the device undergoing activation. The voltage induces currents that establish the electrodes at the potentials required for oxidation according to the present invention.

### DETAILED DESCRIPTION OF THE PRESENTLY PREFERRED EMBODIMENTS

Generally, techniques in accordance with the present invention may be applied to those instances in which an elemental iridium layer (or an iridium alloy) to be activated is electrically coupled to one or more semiconductor junctions. As used herein, a semiconductor junction comprises the boundary between any two or more dissimilarly doped regions within a semiconductor substrate. In a preferred embodiment, a semiconductor junction can be a structure which produces a current in response to excitation. Typically, such excitation comprises illumination by visible light but, in general, may comprise any excitation that generates junction current. Preferably, the junction will comprise a photojunction, but in many instances can be a more complex structure, e.g. one or more CMOS or bipolar transistors or diodes, or other semiconductor structures common in integrated circuits. The junction is formed in a conventional semiconductor substrate, such as monocrystalline silicon although compound semiconductor substrates such as gallium arsenide, silicon carbide, etc. may be equally employed. Additionally, a substrate as used herein may include materials other than semiconductor and dopants such as, for example, various metal layers used to improve electrical connections and/or mechanical adhesion between iridium oxide layers and corresponding semiconductor junctions. For example, a layer of titanium is often used to improve the electrical and mechanical connection between metal electrodes and the underlying semiconductor material.

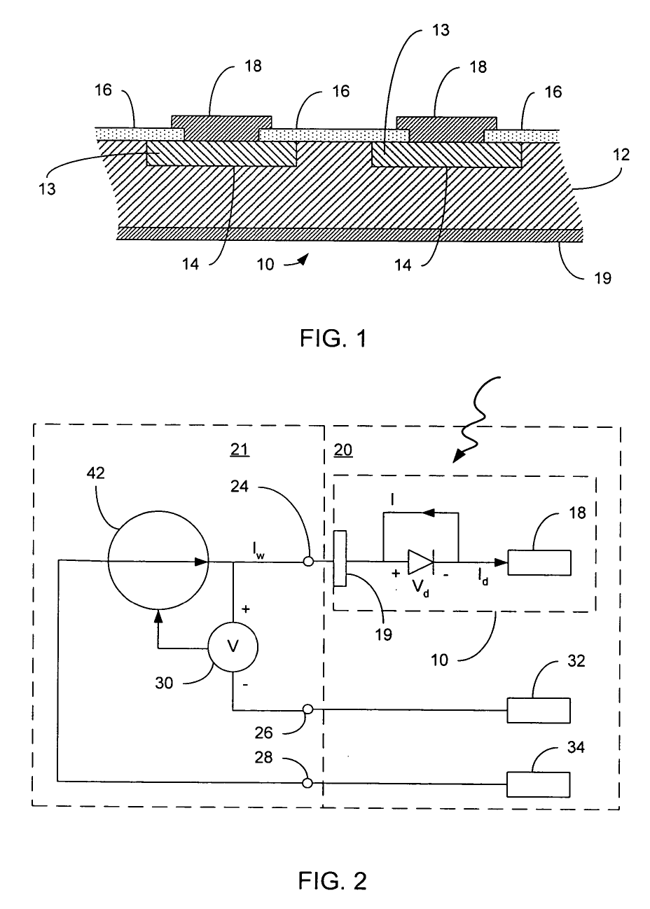

Referring to FIG. 1, a preferred semiconductor structure 10 comprises a semiconductor substrate 12, typically doped silicon having semiconductor junctions 14 formed therein. While the semiconductor substrate is typically silicon, it can be any other semiconductor substrate material, such as gallium arsenide, silicon carbide and the like. In practice, portions of such substrates are doped to form semiconductor junctions with other regions within the substrate. Reflecting a currently preferred embodiment, substrate 12 is shown as a lightly p-doped material, while the regions 13 are n-doped, forming so-called p-n junctions. As know in the art, such p-n junctions 14 exhibit photovoltaic behavior when exposed to certain wavelengths of electromagnetic radiation, e.g., visible or infrared light. In practice, however, the semiconductor junctions 14 could be more complex (comprising, for example, a combination of multiple junctions) with the sole requirement that a sufficiently large junction current can be generated during an activation protocol as discussed in more detail below.

A passivating or insulating layer 16, typically silicon dioxide when the substrate is silicon, is formed over substrate 12, and holes are formed through the layer to allow electrical contact between the underlying substrate and one or more iridium metal electrodes 18 formed on the surface. Preferably, the interface between the iridium electrodes 18 and the underlying semiconductor substrate is annealed prior to activation. Such annealing procedures are known to those having skill in the art and, preferably, comprise heating the substrate and iridium to a temperature in the range of 400° to 450° C. for approximately 30 minutes in a nonreactive environment such as one composed of nitrogen gas. Note that, although the electrodes 18 are illustrated overlying the semiconductor junctions 14, this is not a requirement; in practice, the electrodes 18 only need to be in electrical communication with the semiconductor junctions 14. Other passivating materials, such as amorphous carbon or various polymer-based materials could be equally employed. Formation of the access holes and deposition of the iridium metal electrodes 18 may be accomplished using conventional semiconductor processing techniques. Usually the iridium will be deposited by sputter deposition, but electrochemical deposition, vapor deposition, or other deposition techniques are also possible. As noted above, an additional metallic layer (or layers, not shown) may be employed to improve electrical and/or mechanical performance of the electrodes 18. In the embodiment illustrated in FIG. 1, a common return electrode 19 is provided on a surface opposite the surface upon which the electrodes 18 are placed. In practice, the return electrode 19 may likewise be fabricated through the deposition of elemental iridium that is subsequently oxidized through the activation process described below, although any other commonly used metal may be used. Note that, rather than sharing a common return electrode 19, each semiconductor junction 14 could be electrically isolated from the other junctions and provided with a separate, corresponding return electrode, although this is not preferred.

Generally, the device 10 may comprise any device that requires iridium oxide electrodes to be electrically coupled to one or more semiconductor junctions. In one embodiment of the present invention, the device illustrated in FIG. 1 comprises an implantable medical device, such as a retinal prosthesis or retinal therapeutic device. For example, the device 10 may be designed for intraocular, subretinal implantation for use in treatment for various degenerative retinal diseases. In this case, the device 10 is preferably very thin (typically no more than a few hundred microns thick), with the iridium electrodes 18 likewise correspondingly thin, typically about one hundred nanometers thick.

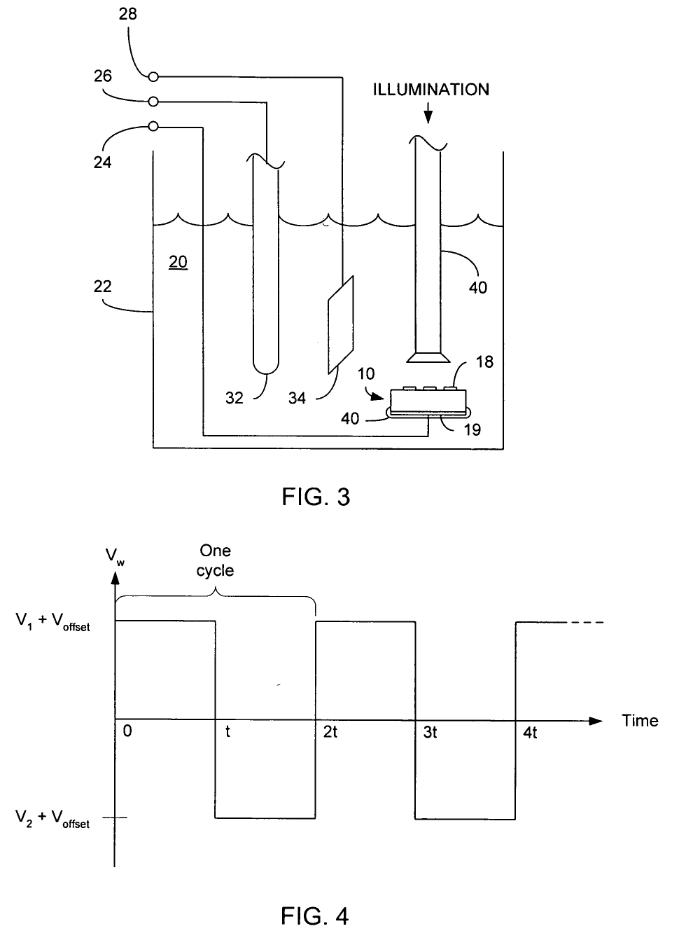

Once the structure 10 shown schematically in FIG. 1 has been fabricated, it is necessary to activate the iridium electrodes 18 to form the desired iridium oxide electrodes. Such activation can be accomplished using an electrochemical cell as illustrated in FIGS. 2 and 3. While the use of the electrochemical cell illustrated in FIGS. 2 and 3 is described with reference to a preferred semiconductor structure 10, it is understood that other semiconductor structures may equally benefit from application of the present invention.

In a preferred embodiment, the semiconductor structure 10 is immersed in an electrolyte bath 20, typically phosphate buffered saline (e.g., 0.3 M Na₂HPO₄) or an equivalent, in an electrolytic cell enclosure 22. The electrochemical cell is generally conventional and operates in conjunction with a potentiostat 21 schematically illustrated as comprising a current source 42 controlled by a voltage-monitoring controller 30. As know in the art, the current source 42 provides a level of working or activation current, I_{w}, necessary to maintain a given voltage between the reference input node 26 and the working input node 24 of the controller 30. Within the electrochemical cell, a reference electrode 32 and a counter electrode 34 (coupled to a counter input node 28 to complete the current path for the current source 42) are used. The reference electrode 32 is typically of the silver/silver chloride (Ag/AgCl) type, though other types may be used. In a conventional setup, a working electrode (coupled to the working output node 24) is connected to or fashioned directly out of the iridium material to be activated. However, given the small size of the iridium electrodes 18 in the preferred embodiment, the working output node 24 is instead coupled to the return electrode 19 and is thereby in electrical communication with the iridium electrodes 18 via the intervening substrate 12 and semiconductor junctions 14. Note that, when activating the iridium electrodes 18 as illustrated in FIGS. 2 and 3, the return electrode 19 is electrically isolated from the electrolyte bath 20. An example of this is illustrated in FIG. 3 by an electrically insulating sealant 40 covering the entirety of the return electrode 19.

To permit the potential of the iridium electrodes 18 to be controlled via currents delivered through the semiconductor junctions 14, an illumination source 40 is provided to direct illumination at the semiconductor structure, particularly the semiconductor junctions 14. In one embodiment, a minimally sufficient level of illumination to allow activation could be employed. Such activation would require relatively complex control of the activation current to compensate for the voltage V_{d} developed by the semiconductor junction 14. As noted above, when a current I_{w} is delivered to the electrode 18 via the intervening semiconductor junction 14, V_{d} will vary with I_{w} in accordance with the characteristics of the junction. Because the activation process requires the voltage between the working electrode (i.e., the iridium electrode 18) and the reference electrode 32 to be accurately controlled, the variations in V_{d} must be taken into account.

More preferably, a substantially higher level of illumination is employed. In this embodiment, the previously mentioned variability and the need for complex current control is substantially eliminated. Typically, the illumination will directly impinge upon that portion of the junction 14 not shadowed by the overlying electrode 18, although a certain portion of the incident illumination may penetrate the electrode 18 depending on the thickness of the electrode 18 and the wavelength of the incident illumination. The illumination preferably induces a photocurrent I_{α} in each semiconductor junction 14 that is sufficiently large to ensure that variations in the voltage across the junctions will be insignificant, thereby permitting the voltage to be treated as a fixed offset. The voltage offset can be measured directly by observing the difference in the open-circuit potential (i.e., the potential under the condition I_{w}=0) at the working node 24 between light and dark conditions. Thus, for example, if it is desired to cycle the electrode 18 between −0.6 V and 0.8 V extremes (with respect to the reference potential), and the difference in open-circuit potential measured at the working node 24 under light versus dark conditions is 0.4 V, the controller 30 is configured to establish the working node 24 at the potential extremes of −0.2 V and 1.2 V.

In a presently preferred embodiment utilizing a silicon photojunction, illumination of the semiconductor junction comprises exposure of the junction to illumination in the range of intensities from 10 to 1000 mW/cm² over a range of visible and near infrared wavelengths that are effective at producing photocurrent, from about 400 nm to 1000 nm. In general, the appropriate choice of intensity and wavelengths will depend upon the properties of the semiconductor junction. In embodiments employing a substantially higher level of illumination, the primary criterion for sufficient illumination is that the induced photocurrent be substantially larger in magnitude than the peak activation current. To the extent that such a condition is achieved, the variations in voltage across the photojunction are reduced.

Referring now to FIG. 4, the voltage being applied at the working output node 24 will typically be delivered in a square wave pattern to induce the desired potentials at the electrode 18. Other patterns, such as linear ramps, may be employed but typically require more time to achieve comparable levels of activation. Referring to the example shown in FIG. 4, the working current will vary within some range as the voltage between the working output node 24 and the reference input node 26 is cycled between the limits V₁+V_{offset} and V₂+V_{offset} where V_{offset} is the voltage offset described above. When using a silver/silver chloride reference electrode, V₁ is typically set to be no greater than 0.9 V and V₂ is typically set to be no less than −0.6 V. In practice, the dwell time, t, at each potential is preferably around 10 seconds leading to a cycle time of approximately 20 seconds. Although the waveform illustrated in FIG. 4 is symmetrical, i.e., the dwell times at the positive and negative potentials are identical, this is not a requirement, and the length of the dwell times need only be sufficient to achieve the desired level of activation. Generally, several hundred cycles as illustrated in FIG. 4 are repeated to fully oxidize the iridium. The number of cycles employed is dictated by the desired charge capacity for the electrode which, in turn, dictates the amount of activation required. In practice, it is usually desirable to employ as little activation as necessary to achieve the required charge capacity, to minimize the mechanical stress induced within the film by the volumetric expansion associated with conversion of the iridium into its oxide form.

The present invention overcomes many of the disadvantages of prior art activation techniques while preserving their benefits. In those instances in which iridium layers are electrically coupled to semiconductor junctions, the present invention provides a technique for activating iridium to provide iridium oxide. Given the particularly beneficial properties of iridium oxide for charge delivery to biological tissues, the present invention may be advantageously employed in the production of devices for electrical stimulation of such tissues.

Although particular embodiments have been disclosed herein in detail, this has been done for purposes of illustration only and is not intended to be limiting with respect to the scope of the appended claims that follow. In particular, it is contemplated by the inventors that various substitutions, alterations, and modifications may be made to the invention without departing from the spirit and scope of the invention as defined by the claims. Other aspects, advantages, and modifications are considered to be within the scope of the following claims.

I claim:

1. A method for oxidizing an iridium layer electrically coupled to a semiconductor junction, the method comprising:
generating a junction current within the semiconductor junction sufficient to permit an activation current to be applied to the iridium layer; and
applying the activation current to the iridium layer through the semiconductor junction to oxidize the iridium.

2. The method of claim 1, wherein the semiconductor junction comprises a diode.

3. The method of claim 1, wherein the semiconductor junction is formed in a doped silicon substrate.

4. The method of claim 1, wherein generating the junction current further comprises applying illumination to the semiconductor junction.

5. A method for forming an iridium oxide electrode that is electrically coupled to a semiconductor junction on a substrate, the method comprising:
depositing an iridium layer on the substrate and in electrical communication with the semiconductor junction;
applying illumination to the semiconductor junction to generate a junction current sufficient to permit an activation current to be applied to the iridium layer; and
applying the activation current to the iridium layer through the semiconductor junction to oxidize the film.

6. The method of claim 5, wherein the semiconductor junction comprises a diode.

7. The method of claim 5, wherein the semiconductor junction is formed in a doped silicon substrate.

8. The method of claim 5, further comprising:
annealing the iridium layer prior to applying the activation current.

9. The method of claim 5, wherein applying the activation current further comprises:
immersing the substrate in an electrochemical cell, wherein the activation current is applied via the electrochemical cell.

10. The method of claim 9, wherein the electrochemical cell comprises a Na₂HPO₄ electrolyte, an Ag/AgCl reference electrode, a counter electrode and a working electrode connected to the substrate in series with the semiconductor junction.

11. In a retinal prosthesis comprising a plurality of microphotodiodes formed in a semiconductor substrate, each of the plurality of microphotodiodes comprising an electrical connection to a corresponding iridium layer formed on a surface of the semiconductor substrate and another electrical connection to a common ground electrode formed on another surface of the semiconductor substrate, a method for activating the iridium layer corresponding to each of the plurality of microphotodiodes, the method comprising:
immersing the substrate in an electrochemical cell;
coupling the common ground electrode to a working electrode of the electrochemical cell;
applying illumination to the plurality of microphotodiodes; and
applying an activation current to the corresponding iridium layer of each of the plurality of microphotodiodes via the working electrode.

12. The method of claim 11, wherein the electrochemical cell comprises a Na₂HPO₄ electrolyte, an Ag/AgCl reference electrode and a counter electrode.
